(19) United States
(12) Patent Application Publication
(10) Pub. No.: US 2024/0108325 A1
(43) Pub. Date: Apr. 4, 2024

(54) METHOD OF FORMING TISSUE FIXATION WITH A PATCH

(71) Applicant: RIVERPOINT MEDICAL, LLC, Portland, OR (US)

(72) Inventor: Nathan COOK, Portland, OR (US)

(21) Appl. No.: 18/372,631

(22) Filed: Sep. 25, 2023

Related U.S. Application Data
(60) Provisional application No. 63/412,138, filed on Sep. 30, 2022.

(51) Int. Cl. A61B 17/04; A61F 2/08

(52) U.S. Cl. CPC A61B 17/0401; A61F 2/0811; A61B 2017/00889; A61B 2017/0464; A61F 2002/087

(57) ABSTRACT

The invention relates to an improved method of double row tissue repair with a graft or patch at the repair site for various applications of arthroscopic surgery requiring tissue to bone attachment. The method includes a patch and one or more knotless, adjustable anchor systems that allow for knotless adjustability of the patch relative to the bone-tissue interface for proper positioning and securement of the patch at the repair site.

### CROSS REFERENCE TO RELATED APPLICATIONS

This application claims the benefit of U.S. Provisional Application No. 63/412,138 entitled “Method of Forming Tissue Fixation with a Patch,” filed Sep. 30, 2022, the disclosure of which are incorporated by reference herein in its entirety.

### TECHNICAL FIELD

The invention relates to apparatus and methods used in arthroscopic surgery. More specifically, the invention relates to an apparatus and methods of attaching tissue to bone using a patch.

### BACKGROUND OF THE INVENTION

Reattachment of soft tissue to bone employing knotless fixation devices are known in the art, particularly for the formation of double row constructs in arthroscopic repairs or arthroscopic rotator cuff repairs with and without the use of patches. However, the reattachment of soft tissue to bone with a patch is difficult due to increased preparation, handling, instrumentation and time which requires the surgeon to have the specialized skills to implement patch augmentation.

### BRIEF SUMMARY OF THE INVENTION

In one embodiment, a method of fixating tissue to bone using a patch comprises the steps of: providing a patch, a first anchor, a second anchor, a third anchor and a fourth anchor, the patch including a body, a first strand, a second strand, a third strand, and a fourth strand, the body including a top portion and a bottom portion, the first strand and the second strand extending outwardly from a top portion of the body, the third strand and the fourth strand extending outwardly from a bottom portion of the body of the patch; deploying the first anchor into a first hole at a first location and the second anchor into a second hole at a second location; securing the first strand of the patch into the first anchor at the first location and the second strand of the patch into the second anchor at the second location, the top portion of the patch is disposed over a portion of the tissue; deploying the third anchor into a third hole at a third location, the third anchor comprising a portion of the second strand and a third strand of the patch, the second strand extending diagonally from a second location to a third location; deploying the fourth anchor into a fourth hole at a fourth location, the fourth anchor comprising a portion of the first strand and a fourth strand of the patch, the first strand extending diagonally from a first location to a fourth location, the second strand and first strand intersect on a top surface of the body of the patch; and positioning the bottom portion of the patch relative to the third and fourth locations, the bottom portion of the patch disposed over a portion of the bone.

In another embodiment, a method of fixating tissue to bone comprises the steps of: providing a patch, a first anchor, a second anchor, a third anchor and a fourth anchor, the patch including a body, a first strand, a second strand, a third strand, and a fourth strand, the body including a top portion and a bottom portion, the first strand and the second strand extending outwardly from a top portion of the body, the third strand and the fourth strand extending outwardly from a bottom portion of the body of the patch; securing the first strand of the patch into the first anchor deployed within a first hole at a first location and the second strand of the patch into the second anchor deployed within a second hole at a second location, the top portion of the patch is disposed over a portion of the tissue; securing the third strand and second strand of the patch into the third anchor deployed into a third hole at a third location, the second strand extending diagonally from a second location to a third location; securing the fourth strand and first strand of the patch into the fourth anchor deployed into a fourth hole at a fourth location, the first strand extending diagonally from a first location to a fourth location, the second strand and first strand intersect on a top surface of the body of the patch; and positioning the bottom portion of the patch relative to the third and fourth locations, the bottom portion of the patch disposed over a portion of the bone.

### BRIEF DESCRIPTION OF THE SEVERAL VIEWS OF THE DRAWINGS

FIG. 1A depicts a front view of a first embodiment of a tensionable patch or graft;

FIGS. 1B-1C depict a front and side view of a second embodiment of a tensionable patch or graft;

FIGS. 1D-1E depict a front and side view of a third embodiment of a tensionable patch or graft;

FIG. 1F depicts a front view of a fourth embodiment of a tensionable patch or graft;

FIG. 1G depicts a front view of a fifth embodiment of a tensionable patch or graft;

FIG. 2 illustrates a flowchart of one embodiment of a method of a double row tissue repair with a graft or patch at the repair site;

FIGS. 3A-3B illustrate flowcharts of alternative embodiments of a method of a double row tissue repair with a graft or patch at the repair site;

FIG. 4 illustrates a flowchart of an alternative embodiment of a method of a double row tissue repair with a graft or patch at the repair site; and

FIGS. 5A-5G illustrate steps of one embodiment of a method of a double row tissue repair with a graft or patch at the repair site.

### DETAILED DESCRIPTION OF THE INVENTION

There exists a need to create a patch and/or method of repair that reduces the handling, instrumentation, and time during arthroscopic surgery. The embodiments herein are directed to an improved tensionable patch system and its respective method of use for arthroscopic repair of soft tissue-to-bone in various fixation configurations. The surgical method allows for the graft or patch to be fixed over the repair site. The desired fixation configurations may allow for more distribution of forces across the surface of the patch, thus distributed onto the surface of the tissue to create more of a “load sharing” between the patch and tissue.

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter.

Numerous specific details are set forth in the following description in order to provide a thorough understanding of the invention. However, the invention may be practiced according to the claims without some or all of these specific details. Furthermore, these embodiments are described with sufficient detail to enable those skilled in the art to practice the invention, and it is to be understood that other embodiments may be employed, and that structural and logical changes may be made without departing from the spirit or scope of the present invention.

Patches or grafts or scaffolds are commonly used during orthopedic surgery for bone-to-soft tissue and/or soft tissue-to-soft tissue, such as rotator cuff repair. For exemplary purposes only, the embodiments will be described below with reference to an arthroscopic rotator cuff repair. However, the invention is not limited to this exemplary embodiment and has applicability to any surgical repair that also involves reattachment of soft tissue-to-soft tissue (for example, reattachment of labral tissue (cartilage) to the glenoid) and/or bone-to-bone.

The inventions disclosed herein provide a knotless patch, graft or scaffold fixation system to enable the repair of the tissue-to-bone bone in one or more desired fixation configurations. In one exemplary embodiment, the desired fixation includes a double-row configuration with an intersecting (“criss-cross”) pattern. The graft or patch is secured into position at the repair site using only the strand tail ends that are affixed to the patch or body by deploying a plurality of suture anchor fixation device assemblies. The one or more patch strands can be used to tension or pull the graft or patch into final position and desired fixation configuration. A portion of the graft or patch comprises a surface that contacts or overlays a portion of the tissue surface and bone surface.

In one embodiment, the tensionable patch system comprises a patch, graft or scaffold 5, 70, 110, 125, 170 and one or more suture fixation delivery devices (not shown). The one or more suture fixation delivery devices may comprise a hard anchor, such as such as an Arthrex “SwivelLock” C anchor or an Arthrex “PushLock” C anchor (Arthrex, Inc., Naples, FL). The one or more suture fixation devices may comprise a soft anchor, such as the Biomet Juggerknot Soft Anchor System (Biomet Sports Medicine, Warsaw, IN). In another embodiment, the tensionable patch system comprises a patch, graft or scaffold 5, 70, 110, 125, 170, a first one or more suture fixation delivery devices (not shown) and a second one or more suture fixation delivery devices (not shown).

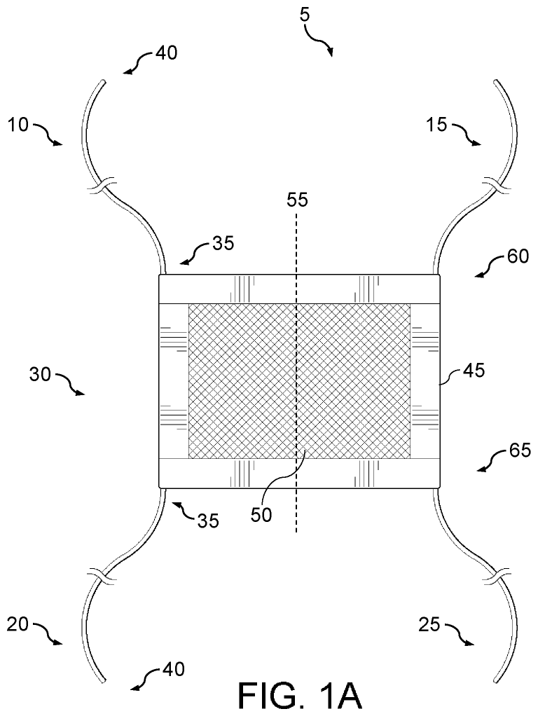

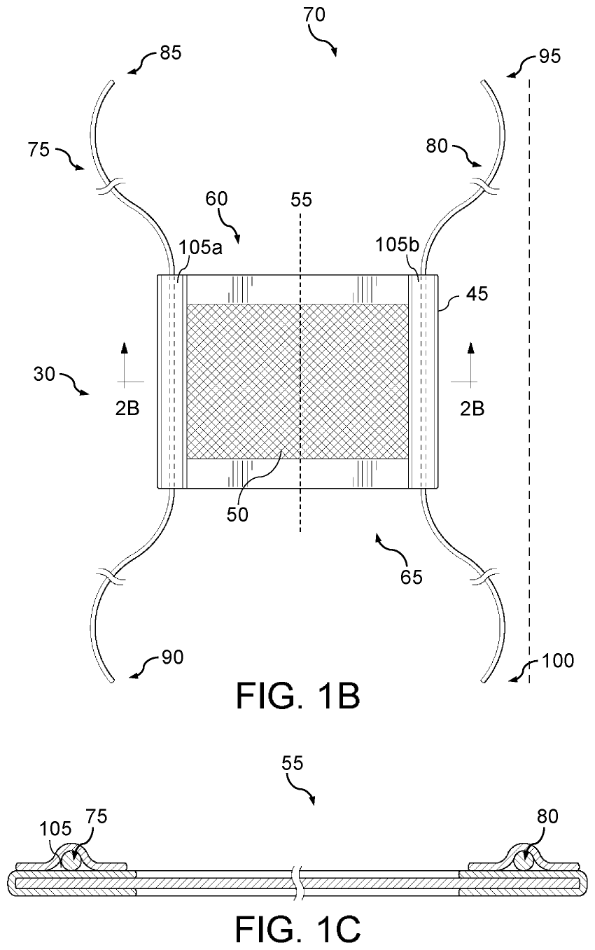

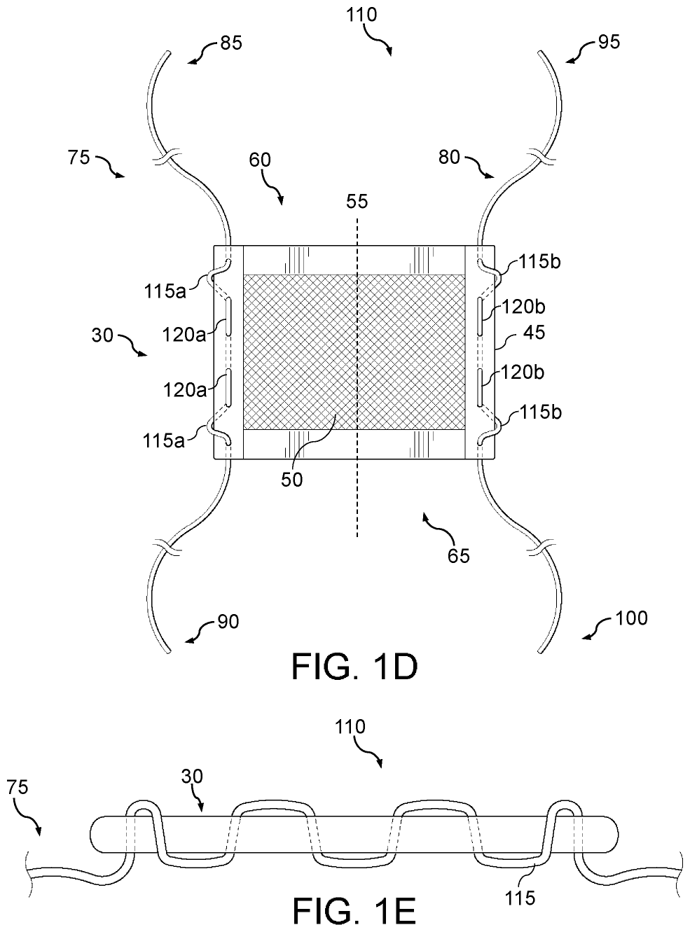

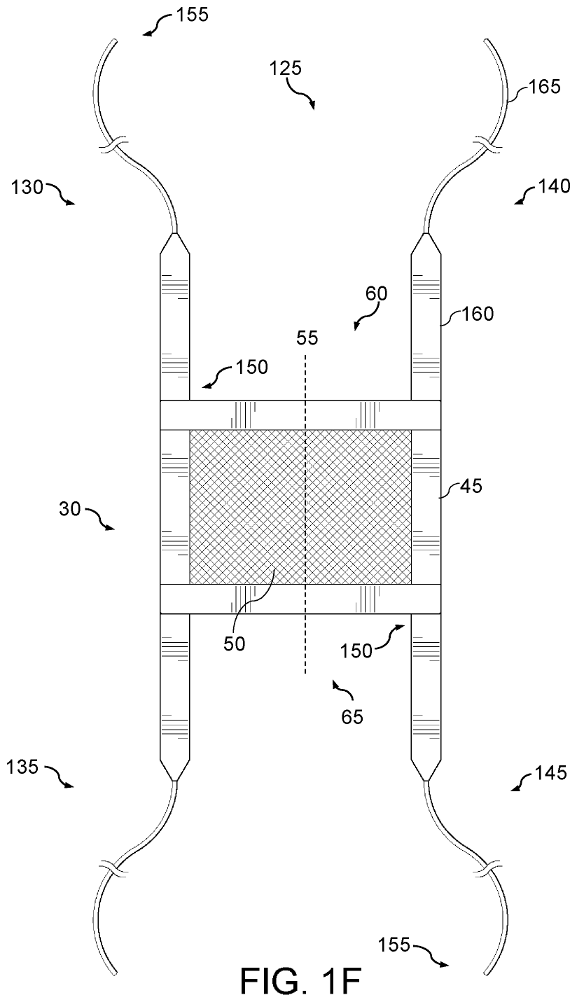

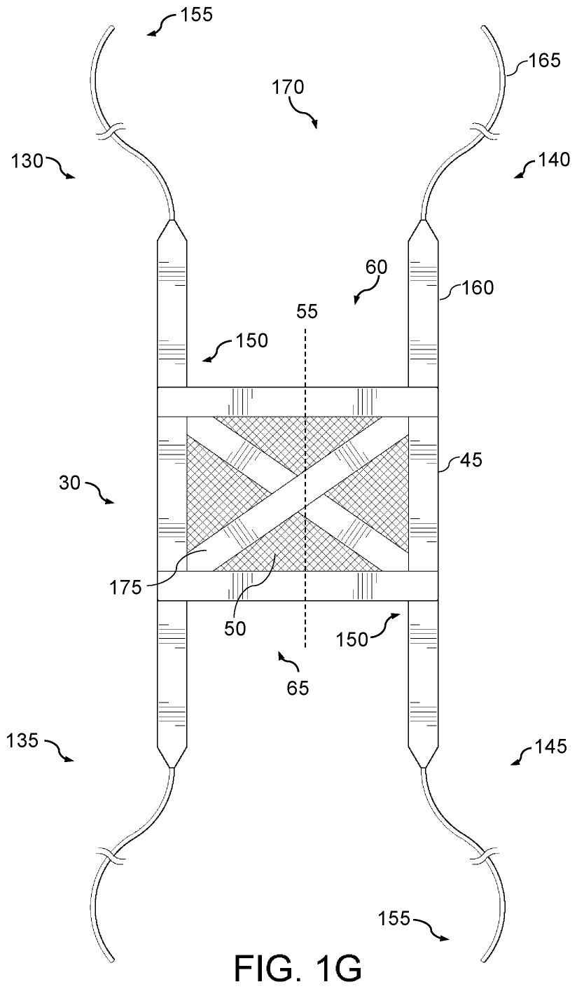

With reference to FIG. 1A, the figure depicts a front plan view of a first embodiment of a tensionable patch or graft 5 of the tensionable patch system. The patch or graft 5 is surgically fixated on top of the tissue-to-bone repair interface to strengthen the repair and aid tissue healing, thereby reducing the likelihood of failure and improving patient outcomes. The patch or graft 5 comprises a body 30 and a plurality of strands or sutures 10, 15, 20, 25. The patch or graft 5 may also comprise a body and at least four sutures or strands 10, 15, 20, 25. Alternatively, the patch or graft 5 comprises a body 30, a first strand or suture 10, a second strand or suture 15, a third strand or suture 20, and/or a fourth strand or sutures 25. Each of the strands 10, 15, 20, 25 comprise a first end 35 and a second end 40 and/or each of the plurality of strands 10, 15, 20, 25 comprise a first end 35 and a second end 40. The body 30 of the patch 5 comprises a border 45, a mesh 50, a longitudinal axis 55, and a shape. The body 30 of the patch 5 may further comprise a first or top portion 60 and/or a second or bottom portion 65.

Each of the strands or each of the plurality of strands 10, 15, 20, 25 of the patch 5 extend from a portion of the body 30 of the tensionable graft or patch. At least two of the four sutures or strands 10, 15 extend from or are coupled to a first portion or top portion 60 of the body 30 of the tensionable graft or patch. The remaining two of the four sutures or strands 20, 25 extend from or is coupled to a second portion or bottom portion 65 of the body 30 of the tensionable patch or graft.

Alternatively, the first end 35 of the first strand 10 and the first end 35 of the second strand 10 is coupled to or extends from a first portion or top portion 60 of the body 30 of the patch 5. The first end 35 of the third strand 20 and the first end 35 of the fourth strand extends from or is coupled to a second or bottom portion 65 of the body 30 of the patch 5. Coupling or fixation may be performed by adhesives, knots, and/or stitch patterns.

With reference to FIG. 1B-1C, the figure depicts a front plan view and a side view of the second embodiment of a tensionable patch or graft 70. In one embodiment, the tensionable patch or graft 70 comprises a body 30, a first strand 75 and a second strand 80. The tensionable patch or graft 70 comprises a body 30 and a plurality of strands or sutures 75, 80. In another embodiment, the tensional patch or graft comprises a body, 30, a first strand end 85, a second strand end 95, a third strand end 90 and a fourth strand end 100. The first strand end 85 and the third strand end 90 may comprise opposing ends of a first single, continuous strand 75, and the second strand end 95 and the fourth strand end 100 comprise opposing ends of a second, single continuous strand 80.

The body 30 of the patch 70 comprises a border 45, a mesh 50, a longitudinal axis 55, and a shape. The body 30 of the patch 70 may further comprise a first or top portion 60, a second or bottom portion 65 and/or one or more lumens or passageways 105a,105b. The lumens or passageways 105a, 105b extend from the top portion of 60 of the body 30 to the bottom portion of the 65 of the body 30. The lumens or passageways 105a, 105b extend longitudinally and/or parallel to the longitudinal axis 55 of the body 30 of the patch 70. The lumens or passageways 105a, 105b are sized and configured to receive a portion of the first strand 75, the second strand 80, and/or each the plurality of strands 75, 80. The lumens or passageways 105a, 105b are sized and configured to receive a portion of the first strand 75, the second strand 80, and/or each the plurality of strands 75, 80 and create a tight-fit or friction-fit to prevent excessive movement of the strands 75, 80 relative to the body 30 of the patch 70. Each of the lumens or passageways 105a, 105b comprise a first end and a second end (not shown).

The first strand 75 comprises a first tail end 85 and a third tail end 90. The second strand 80 comprises a second tail end 95 and a fourth tail end 100. In one embodiment, at least a portion of each of the first strand 75, the second strand 80, and/or each the plurality of strands 75, 80 is threaded or extended through a portion of the body of the graft or patch 70 and/or a portion of the reinforced borders 45. Accordingly, at least a portion of each of the first strand 75, the second strand 80, and/or each the plurality of strands 75, 80 is threaded or extended through a portion of the body of the graft or patch 70 and/or a portion of the reinforced borders 45 to create a first strand end 85, a second strand end 80, a third strand tail end 90 and/or a fourth strand end 100. In another embodiment, at least a portion of each of the first strand 75 is threaded or extended through the lumen or passageway 105a to create a first strand end 85 and a third strand end 90 and at least a portion of the second strand 80 is extended or threading through the lumen or passageway 105b to create a second strand end 95 and a fourth strand end 100. At least a portion of each of the first strand 75, the second strand 80, and/or each the plurality of strands 75, 80 may be coupled or fixated to a portion of lumens or passageways 105a, 105b. At least a portion of each of the first strand 75, the second strand 80, and/or each the plurality of strands 75, 80 may be coupled or fixated to the first end and/or second end of the lumens or passageways 105a, 105b. Coupling or fixation may be performed by adhesives, knots, and/or stitch patterns.

With reference to FIG. 1D-1E, the figure depicts a front plan view and a side view of the third embodiment of a tensionable patch or graft 110. The tensionable patch or graft 110 comprises a body 30, a first strand 75 and a second strand 80. Alternatively, the tensionable patch or graft 110 comprises a body 30 and a plurality of strands or sutures 75, 80. The body 30 of the patch 110 comprises a border 45, a mesh 50, a longitudinal axis 55, and a shape. The body 30 of the patch 110 may further comprise a first or top portion 60, a second or bottom portion 65.

The first strand 75 comprises a first tail end 85 and a second tail end 90. The second strand 80 comprises a third tail end 95 and a fourth tail end 100. In one embodiment, at least a portion of each of the first strand 75, the second strand 80, and/or each the plurality of strands 75, 80 is stitched, threaded or extended through a portion of the body of the graft or patch 70 and/or a portion of the reinforced borders 45 with a stitch pattern. Accordingly, at least a portion of each of the first strand 75, the second strand 80, and/or each the plurality of strands 75, 80 is stitched, threaded or extended through a portion of the body of the graft or patch 110 and/or a portion of the reinforced borders 45 in a stitch pattern to create a first strand end 85, a second strand end 95, a third strand end 90 and/or a fourth strand end 100.

Alternatively, the stitch pattern may include one or more locking stitches or a plurality of locking stitches 115a, 115b. The pattern of stitches may further include a recurring pattern of stitches. The pattern of stitches may further comprise a first type of stitch 115a, 115b and a second type of stitch 120a, 120b. The recurring pattern may include a locking stitch followed by two standard stitches, then ending with a locking stitch. The recurring pattern may further include a first stitch type 115a, a second stitch type 120a, 120b, followed by a first stitch type 115b. The first type of stitch 115a, 115b may include a locking stitch. The second type of stitch 120a, 120b may be a standard stitch or straight stitch. The stitch patterns may include a running stitch, a basting stich, a slip/ladder stich, a whip stitch, a blanket stitch, and/or any combination thereof. The locking stitch may comprise a whip stitch, a blanket stitch, a catch stitch, an overcasting stitch and/or any combination thereof. The standard or straight stitch may comprise a running stitch, a running baste stitch, a backstitch and/or any combination thereof. The first type of stitch 115a, 115b may be disposed at a top portion 60 and/or a bottom portion of the body 30 of the patch 110. The second type of stitch 120a, 120b is disposed between the first type of stitch 115a, 115b. Alternatively, the second type of stitch 120a, 120b is disposed onto a portion of the border 45 and aligns parallel to the longitudinal axis 55 of the body 30. The first type of stitch is disposed onto a portion of the border 45 of the body 30 and may run or align transverse or substantially transverse to the longitudinal axis 55 of the body 30.

In another embodiment, at least a portion of each of the first strand 75 is stitched, threaded or extended through a portion of the border 45 of the body 30 in a stitch pattern to create a first strand end 85 and a third strand end 90 and at least a portion of the second strand 80 is stitched, extended or threading an opposing portion of the border 45 of the body 30 in a stitch pattern to create a second strand end 95 and a fourth strand end 100. At least a portion of each of the first strand 75, the second strand 80, and/or each the plurality of strands 75, 80 may be coupled or fixated to a portion of the border 45 of the body 30. At least a portion of each of the first strand 75, the second strand 80, and/or each the plurality of strands 75, 80 may be coupled or fixated to the first end or portion 60 and/or second end or portion 65 of the body. Coupling or fixation may be performed by adhesives, knots, and/or stitch patterns.

With reference to FIG. 1F-1G, the figure depicts a front plan view of the fourth and fifth embodiment of a tensionable patch or graft 125, 170. The patch or graft 125,170 comprises a body 30 and a plurality of strands or sutures 130, 135, 140, 145. The patch or graft 125, 170 may also comprise a body and at least four sutures or strands 130, 135, 140, 145. Alternatively, the patch or graft 125, 170 comprises a body 30, a first strand or suture 130, a second strand or suture 135, a third strand or suture 140, and/or a fourth strand or sutures 145. Each of the strands 130, 135, 140, 145 comprise a first end 150 and a second end 155 and/or each of the plurality of strands 130, 135, 140, 145 comprise a first end 150 and a second end 155. The body 30 of the patch 125, 170 comprises a border 45, a mesh 50, a longitudinal axis 55, and a shape. The body 30 of the patch 125, 170 may further comprise a first or top portion 60 and/or a second or bottom portion 65. The body 30 of the patch 125, 170 may further comprise one or more reinforcement members 175.

Each of the strands or each of the plurality of strands 130, 135, 140, 145 of the patch 125, 175 extend from a portion of the body 30 of the tensionable graft or patch 125, 170. At least two of the four sutures or strands 130, 140 extend from or are coupled to a first portion or top portion 60 of the body 30 of the tensionable graft or patch 125, 170. The remaining two of the four sutures or strands 135, 145 extend from or is coupled to a second portion or bottom portion 65 of the body 30 of the tensionable patch or graft 125, 170.

Alternatively, the first end 150 of the first strand 130 and the first end 155 of the second strand 135 is coupled to or extends from a first portion or top portion 60 of the body 30 of the patch 125, 170. The first end 150 of the third strand 140 and the first end 150 of the fourth strand 145 extends from or is coupled to a second or bottom portion 65 of the body 30 of the patch 125, 170. Coupling or fixation may be performed by adhesives, knots, and/or stitch patterns. The second end 155 of each of the strands 130, 135, 140, 145 comprises a “free” end. The “free end” may also be referred to as a “tail end.”

In another embodiment, each of the strands 130, 135, 140, 145 comprises a first portion 160 and a second portion 165. The first portion 160 may comprise a first portion width and the second portion 165 comprises a second portion width. The first portion width is greater than the second portion width. The first portion 160 may comprise a different strand than the second portion 165. The first portion 160 may comprise a flat strand, the second portion 165 may comprise a round strand. The first portion 160 may comprise a tab, the second portion 165 may comprise a round strand. The transition of a larger width to a smaller width helps with tension or pulling forces and/or reduces the fixation or coupling point failures.

The plurality of strands, at least four strands, a first strand, a second strand, a third strand, and/or a fourth strand 10, 15, 20, 25, 75, 80, 130, 135, 140, 145 may comprise a color. The colored strands may help the surgeon or user identify each of the strands and how the strands are secured within the bone locations. Alternatively, the “free ends” or the tail ends of the strands 10, 15, 20, 25, 75, 80, 130, 135, 140, 145 may also comprise a color. In one embodiment, the first strand or strand end and second strand or strand end may comprise a first color, and the third strand or strand end and the fourth strand or strand end may comprise a second color. In another embodiment, first strand or strand end and fourth strand or end comprises a first color, and the second strand or strand end and third strand or strand end comprise a second color.

The plurality of strands, at least four strands, a first strand, a second strand, a third strand, and/or a fourth strand 10, 15, 20, 25, 75, 80, 130, 135, 140, 145 may comprise a flat strand, a round strand, a flat-to-round strand, and/or any combination thereof. The plurality of strands, at least four strands, a first strand, a second strand, a third strand, and/or a fourth strand 10, 15, 20, 25, 75, 80, 130, 135, 140, 145 may comprise a textile construction. The textile construction includes a woven, a braided and/or a knitted construction.

The body 30 of the patch or graft 5, 70, 110, 125, 170 may comprise a xenograft, a synthetic, allograft and/or autograft materials. The body 30 of the patch or graft 5, 70, 110, 125, 170 and/or at least a portion of the body 30 of the patch or graft 5, 70, 110, 125, 170 may further comprise a coating. The coating includes at least one or more of an anticoagulant coating, an antimicrobial coating, a polymer coating, a naturally derived coating and/or any combination thereof. The naturally derived coating may include collagen, dermal extracellular matrix, and/or fascia lata.

The body 30 of the graft or patch 5, 70, 110, 125, 170 may further comprise one or more reinforced edges, borders or trims 45 and reinforced members 175. The one or more reinforced trims or borders 45 may be used to provide additional material strength, prevent unraveling and/or fraying. The one or more trims or borders 45 may be positioned or disposed around the perimeter of the body 30 of the graft or patch 5, 70, 110, 125, 170. The one or more trims or borders 45 may further comprise different edge finishes known in the art. The one or more reinforcement members 175 may be positioned or disposed onto a portion of the mesh 50. The one or more reinforcement members 175 may run parallel, transverse, and/or oblique to the longitudinal axis 55 of the body 30. In one embodiment, the one or more reinforcement members 175 may intersect at a desired location.

The strands 10, 15, 20, 25, 75, 80, 130, 135, 140, 145, the body 30 and/or the mesh 50 of the patch 5, 70, 110, 125, 170 comprise a material. The material may comprise a ceramic, polymer and/or metal. The polymer may further include a thermoset or thermoplastic polymer. The material may comprise synthetic or natural suture materials. The material may include a non-biodegradable material or polymer and/or biodegradable material.

The non-biodegradable polymers may comprise nylons, polypropylenes, polyolefins, polyketones, polyamides, and polyesters. Suitable polyolefins include polyethylenes and polypropylenes, especially such polymers of high molar mass like ultra-high molar mass polyethylene (UHMWPE). Suitable polyamides include aliphatic, semi-aromatic and aromatic polyamides, like polyamide 6, polyamide 66 and their copolymers, and poly(phenylene terephthalamide). Suitable polyesters include aliphatic, semi-aromatic and aromatic polyesters, like poly (l-lactic acid) and its copolymers, polyethylene terephthalate (PET), polytrimethylene terephthalate (PTT), polyethylene naphthalate (PEN), polyethylene furanoate (PEF) and liquid crystalline aromatic copolyesters.

The biodegradable materials may comprise a natural or synthetic biodegradable material. Natural biodegradable polymers include chitosan, silk fibroin, fibrinogen, collagen and hyaluronic acid. Synthetic biodegradable polymers include poly(ε-caprolactone) (PCL), polydioxanone (PDA), PLA, PGA, copolymer PLGA, polytrimethylene carbonate (PTMC), and/or poly(p-dioxanone) (PDO). These materials have been proven to be biocompatible and have a controlled degradation rate, and their degradation products in-vivo have no toxic effects on tissues.

Semi-synthetic or bio-derived biocompatible polymers include materials like derivates of proteins and polysaccharides, such as cellulose. Synthetic biocompatible polymers include materials like poly (meth) acrylates, polyolefins, vinyl polymers, fluoropolymers, polyesters, polyamides, polysulfones, polyacrylics, polyacetals, polyimides, polycarbonates, polyethylenes, polyurethanes, including copolymers, compounds and blends thereof. Such synthetic polymers may be based on natural compounds like amino acids and/or on synthetic monomers.

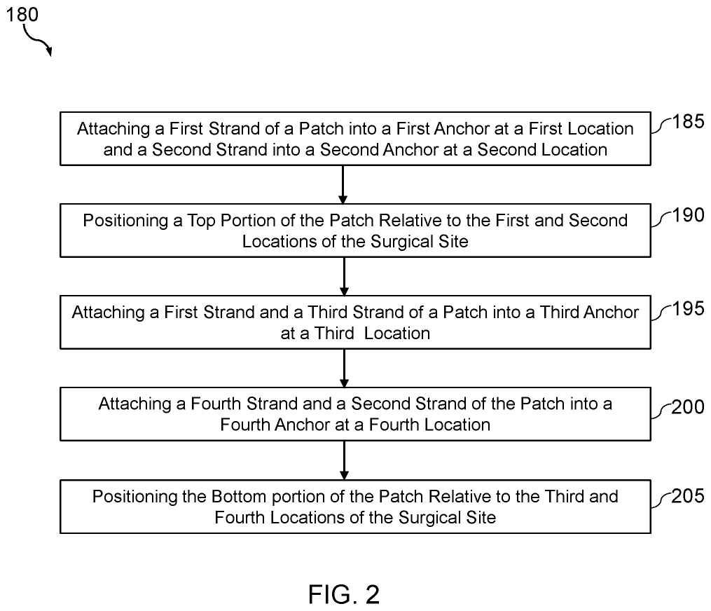

With reference to FIG. 2, the flowchart illustrates a first embodiment of a method of fixating soft tissue to bone comprises using a graft or patch 180 comprises the steps of: attaching a first strand of a patch into a first anchor at a first location and a second strand of a patch into a second anchor at a second location 185; positioning a top portion of the patch relative to the first and second locations of the surgical site 190; attaching a first strand and a third strand of a patch into a third anchor at a third location 195; attaching a second strand and a fourth strand of the patch into a fourth anchor at a fourth location 200; and positioning the bottom portion of the patch relative to the third and fourth locations of the surgical site 205.

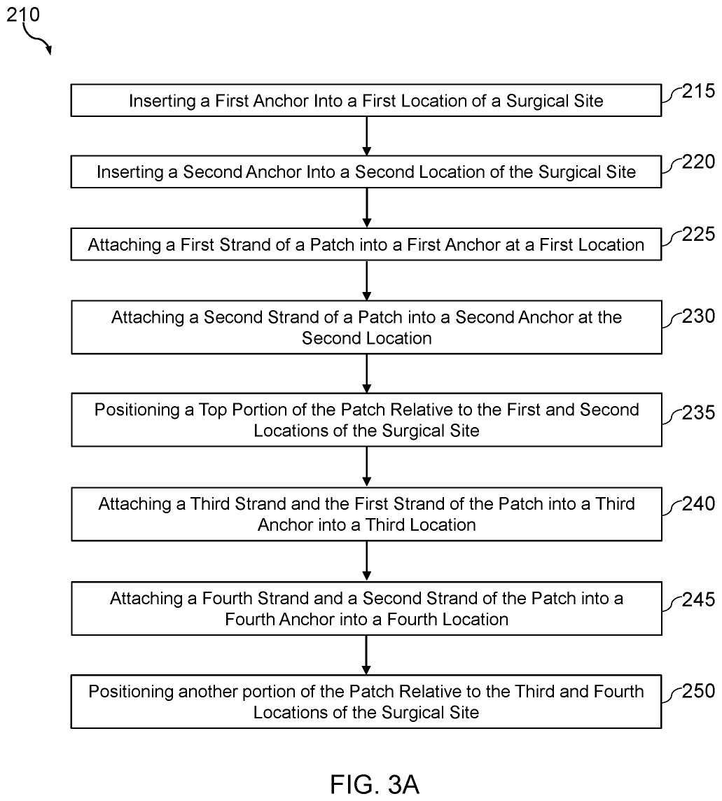

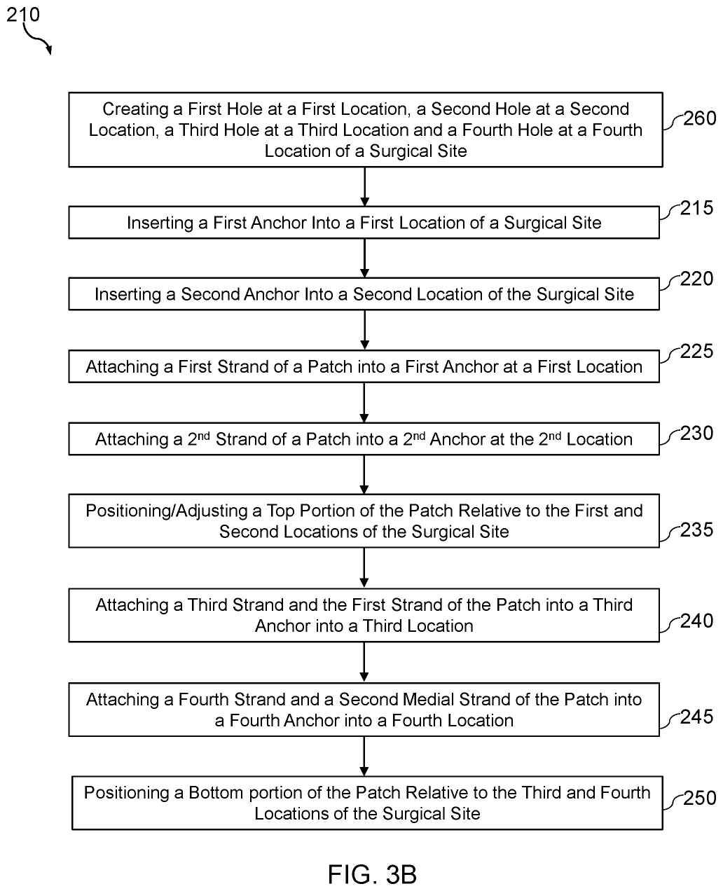

With reference to FIGS. 3A-3B, the flowchart illustrates a second embodiment of a method of fixating soft tissue to bone comprises using a graft or patch 210 comprises the steps of: inserting a first anchor into a first location at a surgical site 215; inserting a second anchor into a second location at the surgical site 220; attaching a first strand of a patch into the first anchor at the first location 225; attaching a second strand of the patch into the second anchor at the second location 230; positioning a top or first portion of the patch relative to the first and second locations 235; inserting or deploying a third strand and the first strand of a patch into a third anchor at a third location 240; inserting or deploying a fourth strand and a second strand of the patch into a fourth anchor at a fourth location 245; and positioning a bottom or second portion of the patch relative to the third and fourth locations 250. The method may further comprise the step of passing the first suture strands of the first anchor and the second suture strands of the second anchor through a portion of the tissue (not shown). The method may further comprise the step of trimming the first, second, third and fourth suture strands (not shown). The method may further comprise the step of creating a first hole in a first location, a second hole in a second location, a third hole in a third location and a fourth hole in a fourth location 260 as shown in FIG. 3B.

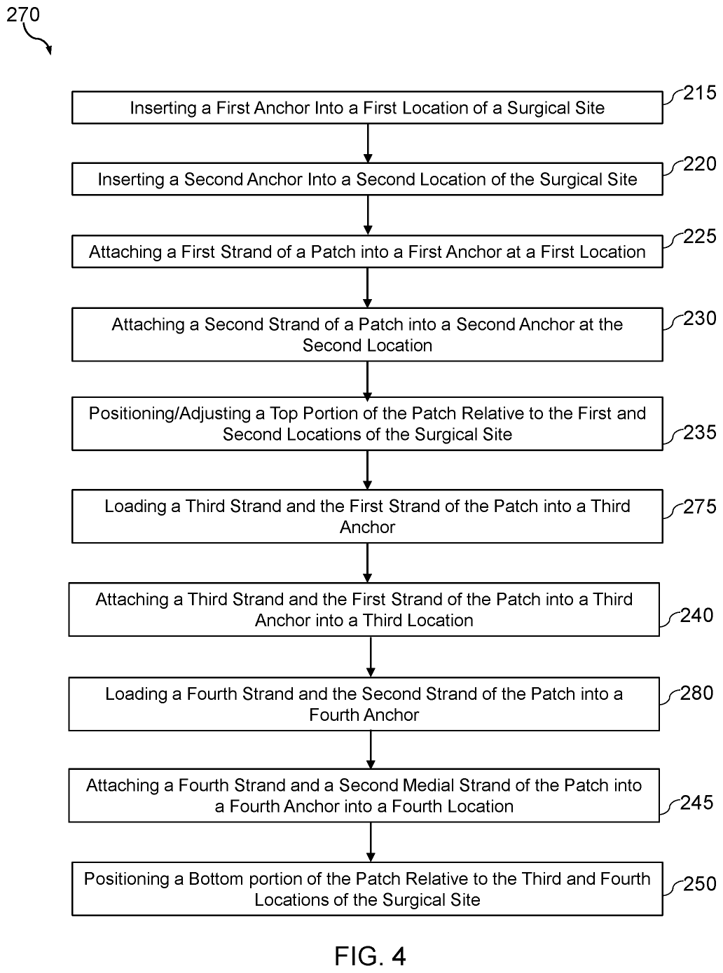

With reference to FIG. 4, the flowchart illustrates a second embodiment of a method of fixating soft tissue to bone comprises using a graft or patch 270 comprises the steps of: inserting a first anchor into a first location at a surgical site 215; inserting a second anchor into a second location at the surgical site 220; attaching a first strand of a patch into the first anchor at the first location 225; attaching a second strand of the patch into the second anchor at the second location 230; positioning a top or first portion of the patch relative to the first and second locations 235; loading a first strand and a third strand of the patch into a third anchor 275; inserting or deploying a third strand and the first strand of a patch into a third anchor at a third location 240; loading a second strand and a fourth strand of the patch into a fourth anchor 280; inserting or deploying a fourth strand and a second strand of the patch into a fourth anchor at a fourth location 245; and positioning a bottom or second portion of the patch relative to the third and fourth locations 250. The method may further comprise the step of passing the first suture strands of the first anchor and the second suture strands of the second anchor through a portion of the tissue (not shown). The method may further comprise the step of trimming the first, second, third and fourth suture strands (not shown). The method may further comprise the step of creating a first hole in a first location, a second hole in a second location, a third hole in a third location and a fourth hole in a fourth location 260 as shown in FIG. 3B.

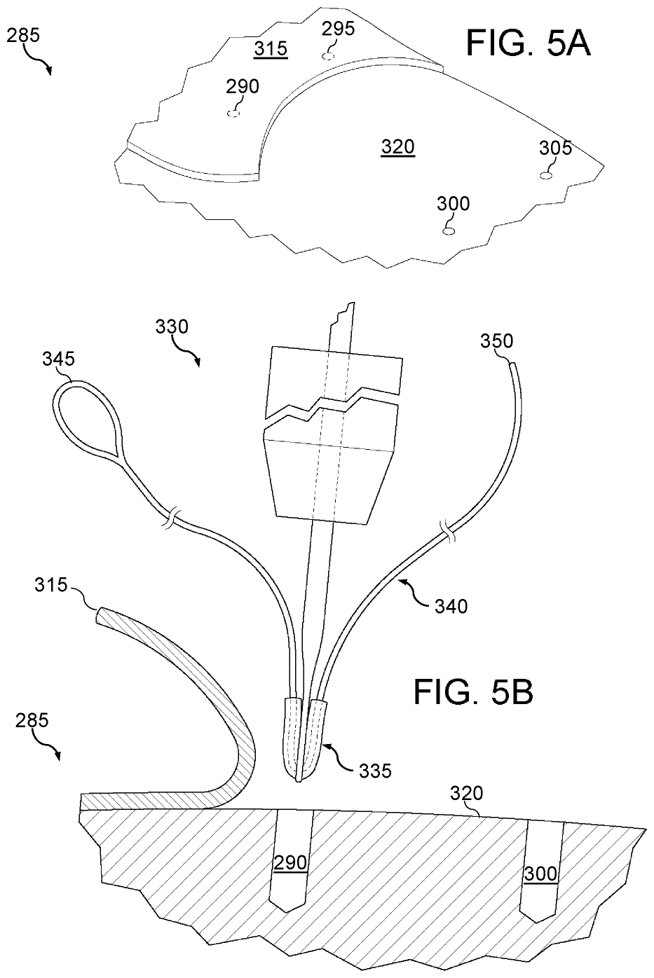

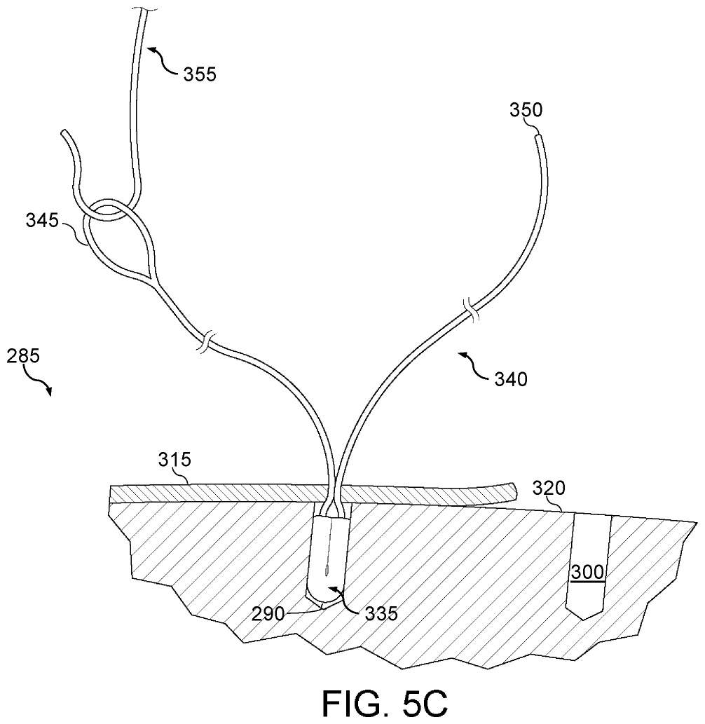

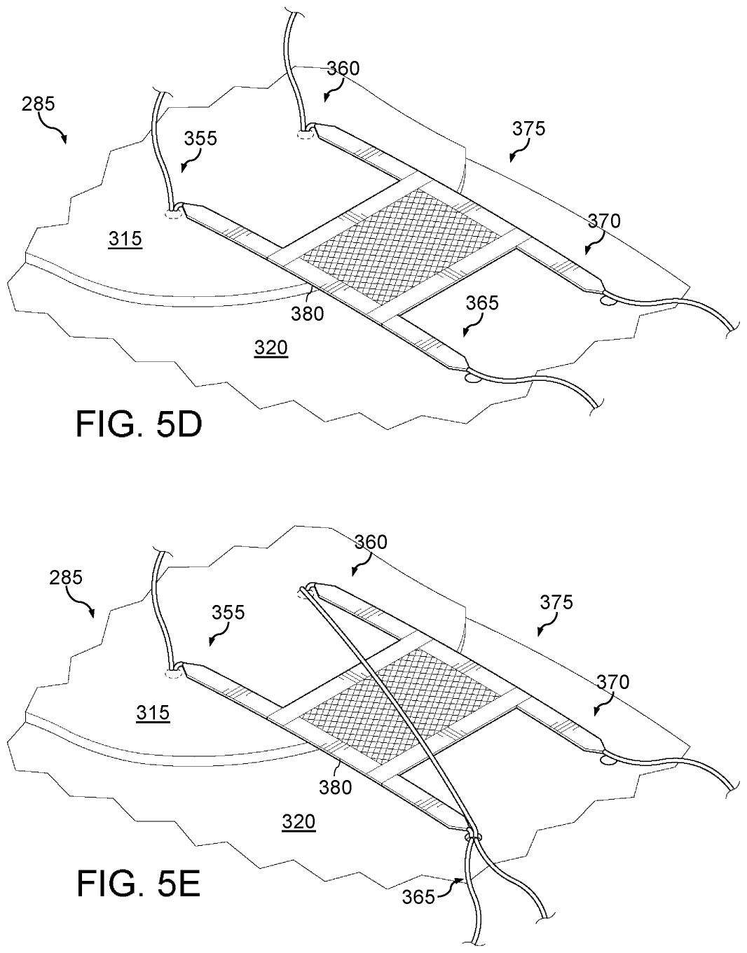

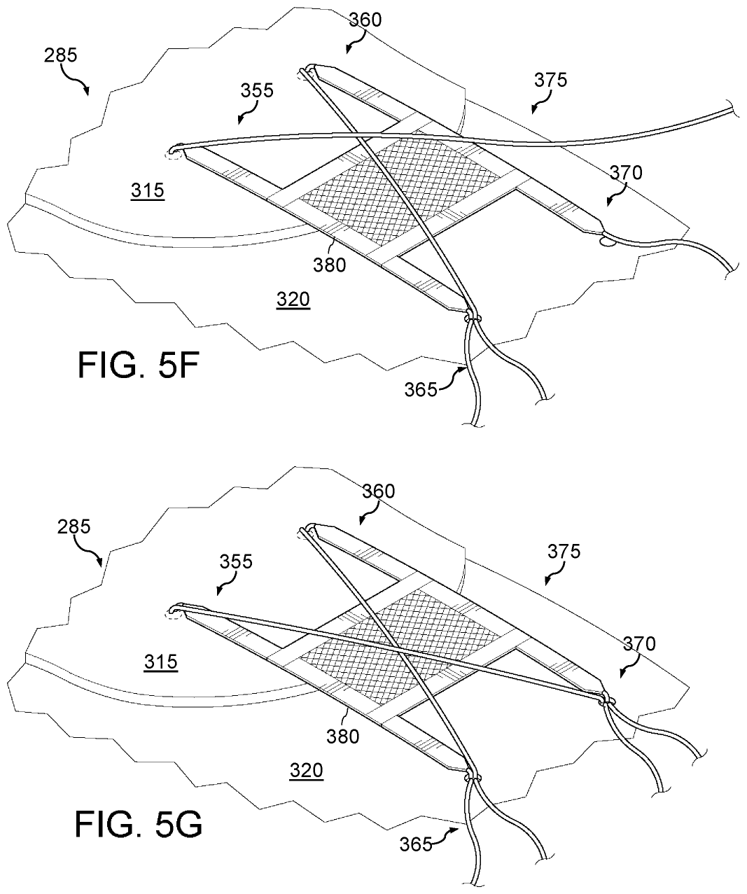

With reference to FIG. 5A, the figure schematically illustrates the step of creating one or more holes or pilot holes disposed or positioned onto one or more locations 260 (see FIG. 3B). In one embodiment, a method of fixating soft tissue to bone comprises using a graft or patch 180, 210, 270 comprises providing a patch 375 and one or more suture fixation delivery devices with one or more anchors or anchoring elements; and the step of creating one or more holes 290, 295, 300, 305 at the surgical site 285. The patch 375 comprises a body 380, first strand 355, a second strand 360, a third strand 365 and a fourth strand 370. The one or more suture fixation delivery devices with one or more anchors or anchoring elements may comprise a first suture fixation delivery device 330 with a first anchor 335, a second suture anchor fixation delivery device (not shown) with a second anchor (not shown), a third suture anchor fixation delivery device (not shown) with a third anchor (not shown), and a fourth suture anchor delivery device (not shown) with a fourth anchor (not shown).

The step of creating a first hole in a first location 290, a second hole in a second location 295, a third hole in a third location 300, and/or a fourth hole in a fourth location 305 at a surgical site 285 can be accomplished with tools and techniques known in the art. Each of the one or more holes and locations 290, 295, 300, 305 are spaced apart. Each of the one or more holes and/or locations 290, 295 are spaced apart and parallel or substantially parallel to the adjacent one or more holes and/or locations 300, 305. At least a portion of the one or more holes, a first hole at a first location 300 and a second hole at a second location 305 are positioned underneath a portion of the tissue 315 (e.g., such as a rotator cuff). At least a portion of the one or more holes, the third hole in a third location 300, and/or the fourth hole at a fourth location 305 are positioned adjacent to the tissue 315 (e.g., such as a rotator cuff) and onto a bone surface 320.

In another embodiment, a first hole is placed in first location 290, a second hole is positioned or placed in a second location 295, a third hole is positioned in a third location 300 and a fourth hole is positioned in fourth location 305. The first hole at the first location 290 is spaced apart from the second hole at a second location 295. The first hole at the first location 290 is spaced apart and axially aligned with the second hole at the second location 295. The third hole at the third location 300 is spaced apart from the fourth hole at the fourth location 305. The third hole at the third location 300 is spaced apart and parallel or substantially parallel from the fourth hole at a fourth location 305. The first hole at the first location 290 and second hole at a second location 295 is spaced apart and parallel or substantially parallel to the third hole at a third location 300 and fourth hole at a fourth location 305.

A first suture fixation delivery device 330 should be obtained. The first fixation delivery device 330 comprises an anchoring element 335 and/or a shuttle 340. The anchoring element 335 includes a soft anchor or a hard anchor. The shuttle 340 includes a shuttle loop 345 and a free end 350. The first suture fixation delivery device 330 may be inserted into the first hole at the first location 290 to deploy the first anchoring element or anchor 335 into the first hole at the first location 290 and the shuttle 340.

At least a portion of the shuttle 340 should extend through or be passed through the tissue 315 as shown in FIG. 5C. The passing of the shuttle 340 may be performed by using a suture passer (not shown), e.g., a BiPass Suture Passer (Biomet Sports Medicine, Warsaw, IN) to allow access to shuttle loop 345 and the shuttle free end 350 over or through the surface of the tissue 315. The suture passer may puncture the tissue 315 to create a tissue hole that may be concentric or axially aligned to the first hole at the first location 290 to enable the shuttle loop 345 and the shuttle free end 350 to exit out the tissue hole.

Once the shuttle loop 345 and the shuttle free end 350 exits out the tissue hole, the first strand 355 of the patch 375 should be attached or secured into a first anchor or anchoring element 335. The free end of the first strand 355 will be passed through the shuttle loop 345 and the shuttle free end 350 is pulled until the shuttle is removed from the first anchor or anchoring element 335 to enable access to the free end or tail end of the first strand 355. At least a portion of the first strand 355 extends through a lumen of the first anchor or anchoring element 335. The second anchor or anchoring element is deployed into the second location, the second anchor having a second shuttle (not shown).

The steps of deploying a second anchor into the second hole at the second location 295 with a second suture fixation delivery device (not shown); passing the second shuttle of the second through the tissue; and/or attaching or securing the second strand into or through the second anchor in a second hole at a second location 295 should be repeated as shown in FIG. 5D.

The first strand 355 of the patch 375 should be positioned relative to the first hole at the first location 290 and the second strand 360 of the patch should be positioned relative to the second hole at the second location 295 as shown in FIG. 5D. The first free end of first strand 355 of the patch 375 and the second free end or tail end 295 of the patch 375 may be pulled or tensioned to position a first portion or top portion of the patch 375 or the body of the patch 375 relative to the first hole at the first location 290 and the second hole at the second location 295. The first portion or top portion of the patch 375 contacts and/or overlaps the portion of a top surface of the tissue 315 and a second portion or bottom portion of the patch 375 contacts and/or overlaps a portion of the bone 320 or tissue 315. A first portion or top portion of the body 380 of the patch 375 contacts and/or overlaps the portion of the top surface of the tissue 315 and a second portion or bottom portion of the body 380 of the patch 375 contacts and/or overlaps the portion of the top surface of a bone 320 or tissue 315.

A third suture fixation delivery device and a fourth suture fixation delivery device (not shown) should be obtained. The third and fourth fixation delivery device comprises a third and fourth anchor or anchoring element. The anchor or anchoring element includes a hard anchor or soft anchor. The hard anchor having an eyelet. A free end or portion of the third strand 365 and a second strand 360 of the patch 375 can be loaded into the eyelet of the third anchor by using a snare or shuttle tool. The snare may capture the free ends of second strand 360 of the patch 375 and a third strand 365 of the patch 375 to be passed or extended through the eyelet of the third anchor of the third anchor fixation device. The third suture fixation delivery device (not shown) may be inserted into the third hole at the third location 300 to deploy the third anchoring element or anchor 335 as shown in FIG. 5E. The free ends of the second 360 and third strands 365 of the patch 375 may be tensioned lightly to facilitate control the overall tension of the patch 375. The second strand 360 extends diagonally from the second hole at the second location 295 to the third hole at the third location 300. The diagonal orientation may comprise 15 degrees to 60 degrees and/or at least 45 degrees.

The steps of loading or passing the fourth strand 370 and the first strand 360 through an eyelet of the fourth suture fixation delivery device; deploying the fourth anchor into the fourth hole at the fourth location 305 with the fourth suture fixation delivery device (not shown); and/or attaching or securing the fourth strand 365 and the first strand 355 into or through the fourth anchoring element or anchor in the fourth hole at a fourth location 305 should be repeated as shown in FIG. 5F-5G. Such insertion allows a portion of the first strand 355 and the second strand 360 to contact a portion of the top surface of the patch 375 or a top surface of the body 380 of the patch 375 and extend diagonally. The first strand 355 and the second strand 360 intersect to create a double row fixation with a criss-cross or intersecting pattern as shown in FIG. 5G. The intersection of the first strand 355 and the second strand 360 provides additional reinforcement to the center of the body 380 of the patch. The diagonal orientation may comprise 15 degrees to 60 degrees and/or at least 45 degrees.

Alternatively, the first strand 355 and the third strand 365 may be attached or secured to the third anchor in the third hole at a third location 300 and the second strand 360 and the fourth strand 370 may be attached or secured to the fourth anchor in a fourth hole at the fourth location 305 to create a double row fixation with a parallel pattern. The first strand 355 and the second strand 360 will be parallel to each other.

The method may further comprise the step of positioning or adjusting the second portion or bottom portion of the patch 375 relative to the third hole at the third location 300 and the fourth hole at the fourth location 305. The free ends of each of the strands within the third hole at the third location 300 and the fourth hole at the fourth location 305 may be tensioned, pulled or adjusted until a second portion or bottom portion of the graft or patch is flush with a portion of the bone surface 320 or tissue surface 315.

The first anchor of the first anchor fixation device may comprise a same anchor compared to the second anchor of the second anchor fixation device. The first anchor of the first anchor fixation device may comprise a different anchor than the second anchor of the second anchor fixation device. The third anchor of the third anchor fixation device may comprise the same anchor as the fourth anchor of the fourth anchor fixation device. The third anchor of the third anchor fixation device may comprise a different anchor than the fourth anchor of the fourth anchor fixation device. The first anchor of the first anchor fixation device comprises a same anchor as the third anchor of the third fixation device. The first anchor of the first anchor fixation device comprises a different anchor than the third anchor of the third fixation device. The second anchor of the second anchor fixation device comprises a same anchor as the fourth anchor of the fourth fixation device. The second anchor of the second anchor fixation device comprises a different anchor than the fourth anchor of the fourth fixation device. The first and second anchors comprise a same or different anchor compared to the third and fourth anchors.

In another embodiment, the first plurality of anchors of a first plurality of anchor fixation devices comprise a different anchor compared to the second plurality of anchors of a second plurality of anchor fixation devices. The first plurality of anchors of a first plurality of anchor fixation devices comprise a same anchor compared to the second plurality of anchors of a second plurality of anchor fixation devices.

### Additional Embodiments

A method of fixating soft tissue to bone comprises the steps of: deploying a first plurality of knotless fixation devices into a first plurality of bone locations; attaching or securing a first plurality of strands of a tensionable patch or graft to the first plurality of bone locations; positioning a first portion of the tensionable patch or graft relative to the first plurality of bone locations by tensioning the first plurality of strands of the tensionable patch; loading the second plurality of strands of the tensionable patch or graft to a second plurality of knotless fixation devices; deploying a second plurality of knotless fixation devices with a portion of a second plurality of strands of the tensionable patch or graft into a second plurality of bone locations; and positioning a second portion of the tensionable patch or graft relative to the second plurality of bone locations by tensioning the second plurality of strands of the tensionable patch or graft.

The step of positioning a second portion of the tensionable patch or graft relative to the second plurality of bone locations by tensioning the second plurality of strands of the tensionable patch or graft comprises creating a portion of the first plurality of strands and a portion of the second plurality of strands intersect. The first plurality of knotless fixation devices comprises a soft or hard anchor. The second plurality of knotless fixation devices comprises a soft or hard anchor. Alternatively, the first plurality of knotless fixation devices comprises a soft anchor and the second plurality of knotless fixation devices comprises a hard anchor. In another embodiment, the tensionable patch or graft comprises a coating. The coating comprises an antimicrobial coating, an anticoagulant coating, an antioxidant coating, a polymer coating and/or any combination thereof.

I/We claim:

1. A method of fixating tissue to bone using a patch comprises the steps of:
providing a patch, a first anchor, a second anchor, a third anchor and a fourth anchor, the patch including a body, a first strand, a second strand, a third strand, and a fourth strand, the body including a top portion and a bottom portion, the first strand and the second strand extending outwardly from a top portion of the body, the third strand and the fourth strand extending outwardly from a bottom portion of the body of the patch;
deploying the first anchor into a first hole at a first location and the second anchor into a second hole at a second location;
securing the first strand of the patch into the first anchor at the first location and the second strand of the patch into the second anchor at the second location, the top portion of the patch is disposed over a portion of the tissue;
deploying the third anchor into a third hole at a third location, the third anchor comprising a portion of the second strand and a third strand of the patch, the second strand extending diagonally from a second location to a third location;
deploying the fourth anchor into a fourth hole at a fourth location, the fourth anchor comprising a portion of the first strand and a fourth strand of the patch, the first strand extending diagonally from a first location to a fourth location, the second strand and first strand intersect on a top surface of the body of the patch; and
positioning the bottom portion of the patch relative to the third and fourth locations, the bottom portion of the patch disposed over a portion of the bone.

2. The method of claim 1, wherein the first strand and the third strand comprise opposing ends of a first single, continuous strand, and the second strand and fourth strand comprise opposing ends of a second single, continuous strand.

3. The method of claim 2, wherein at least a portion of the first single, continuous strand extends through a first lumen disposed on a portion of the body of the patch on a first side and at least a portion of the second single, continuous strand extends through a second lumen disposed on a portion of the body of the patch on a second side.

4. The method of claim 2, wherein at least a portion of the first single, continuous strand extends through a portion of the body of the patch on a first side in a first stitch pattern and at least a portion of the second single, continuous strand extends through a portion of the body of the patch on a second side in a second stitch pattern.

5. The method of claim 4, wherein the first and second stitch pattern comprises at least one locking stitch and at least one straight stitch.

6. The method of claim 1, wherein the first anchor and the second anchor comprise a soft anchor.

7. The method of claim 6, wherein the third anchor and the fourth anchor comprise a hard anchor.

8. The method of claim 1, wherein the patch comprises a coating, the coating comprises an antimicrobial coating, an anticoagulant coating, an antioxidant coating, a polymer coating and/or any combination thereof.

9. The method of claim 1, wherein the first strand and the second strand comprise opposing ends of a single, continuous strand.

10. A method of fixating tissue to bone comprises the steps of:
providing a patch, a first anchor, a second anchor, a third anchor and a fourth anchor, the patch including a body, a first strand, a second strand, a third strand, and a fourth strand, the body including a top portion and a bottom portion, the first strand and the second strand extending outwardly from a top portion of the body, the third strand and the fourth strand extending outwardly from a bottom portion of the body of the patch;
securing the first strand of the patch into the first anchor deployed within a first hole at a first location and the second strand of the patch into the second anchor deployed within a second hole at a second location, the top portion of the patch is disposed over a portion of the tissue;
securing the third strand and second strand of the patch into the third anchor deployed into a third hole at a third location, the second strand extending diagonally from a second location to a third location;
securing the fourth strand and first strand of the patch into the fourth anchor deployed into a fourth hole at a fourth location, the first strand extending diagonally from a first location to a fourth location, the second strand and first strand intersect on a top surface of the body of the patch; and
positioning the bottom portion of the patch relative to the third and fourth locations, the bottom portion of the patch disposed over a portion of the bone.

11. The method of claim 10, wherein the first anchor and second anchor comprise a soft anchor.

12. The method of claim 10, wherein the third anchor and fourth anchor comprise a hard anchor.

13. The method of claim 10, wherein the patch comprises a coating, the coating comprises an antimicrobial coating, an anticoagulant coating, an antioxidant coating, a polymer coating and/or any combination thereof.

14. The method of claim 10, wherein each of the first strand, second strand, third strand and fourth strand comprise a flat-to-round strand.

15. The method of claim 10, wherein each of the first strand, second strand, third strand and fourth strand comprise a round strand.

16. The method of claim 10, wherein the first strand and the third strand comprise opposing ends of a first single, continuous strand, and the second strand and fourth strand comprise opposing ends of a second single, continuous strand.

17. The method of claim 16, wherein at least a portion of the first single, continuous strand extends through a first lumen disposed on a portion of the body of the patch on a first side and at least a portion of the second single, continuous strand extends through a second lumen disposed on a portion of the body of the patch on a second side.

18. The method of claim 16, wherein at least a portion of the first single, continuous strand extends through a portion of the body of the patch on a first side in a first stitch pattern and at least a portion of the second single, continuous strand extends through a portion of the body of the patch on a second side in a second stitch pattern.

19. The method of claim 18, wherein the first and second stitch pattern comprises at least one locking stitch and at least one straight stitch.

20. The method of claim 10, wherein the first strand and the second strand comprise opposing ends of a single, continuous strand.
